(19) United States
(12) Patent Application Publication
(10) Pub. No.: US 2011/0129555 A1
(43) Pub. Date: Jun. 2, 2011

(54) BURGLARPROOF SPRAYING LIQUID

(76) Inventor: TIAN-CHUAN LIU, Hsinchu County (TW)

(21) Appl. No.: 12/628,219

(22) Filed: Dec. 1, 2009

(51) Int. Cl. A61K 36/31; A61P 43/00

(52) U.S. Cl. 424/755

(57) ABSTRACT

A burglarproof spraying liquid comprising: water; cayenne pepper having a content of about 0.1% to 30% of water (weight percentage); mustard having a content of about 0.1% to 30% of water (weight percentage); natural nano-bacteriostat having a content of about 0.1% to 30% of water (weight percentage); emulsion having a content of about 0.1% to 30% of water (weight percentage); and potassium sorbate having a content of about 0.1% to 30% of water (weight percentage). The liquid of the present invention is harmless to human bodies, while a burglar sprayed by the liquid will strongly feel uneasy and thus be motionless for a longer time, thus therefore, other people can capture the burglar easily.

### FIELD OF THE INVENTION

The present invention relates to burglarproof, and in particular to burglarproof spraying liquid which containing water, cayenne pepper, mustard, natural nano-bacteriostat, emulsion, and potassium sorbet. The liquid of the present invention is harmless to human bodies, while a burglar sprayed by the liquid will strongly feel uneasy and thus be motionless for a longer time, and therefore, other people can capture the burglar easily.

### BACKGROUND OF THE INVENTION

Currently, robberies occur very frequently, especially in banks, supermarkets, convenience stores, etc. Generally, robberies occur in very short time period. The robs escape from the scenes quickly. It is difficult to catch the robbers at the occurring time. However, when the robbers escape from the scene, it will increase the difficulty to catch them.

In one prior art, electric shock is applied to an intruder to attack him so that the intruder feel ache and thus is motionless in a time period. People can catch the intruder at this moment.

However, this prior art way is not a preferred way for burglar proof, since the electric shock is only applied at a very small point. The burglar is very easy to dodge away from the spot of the electric shock.

Thus there is an eager demand for a novel device which can resolve the above mentioned problem.

### SUMMARY OF THE INVENTION

Accordingly, the primary object of the present invention is to provide burglarproof spraying liquid which containing water, cayenne pepper, mustard, natural nano-bacteriostat, emulsion, and potassium sorbet. The liquid of the present invention is harmless to human bodies, while a burglar sprayed by the liquid will strongly feel uneasy and thus be motionless for a longer time, thus therefore, other people can capture the burglar easily.

To achieve above objects, the present invention provides a burglarproof spraying liquid comprising: water; cayenne pepper having a content of about 0.1% to 30% of water (weight percentage); mustard having a content of about 0.1% to 30% of water (weight percentage); natural nano-bacteriostat having a content of about 0.1% to 30% of water (weight percentage); emulsion having a content of about 0.1% to 30% of water (weight percentage); and potassium sorbate having a content of about 0.1% to 30% of water (weight percentage).

The various objects and advantages of the present invention will be more readily understood from the following detailed description when read in conjunction with the appended drawing.

### BRIEF DESCRIPTION OF THE DRAWINGS

FIG. 1 shows the component and ratio of the spraying liquid of the present invention.

FIG. 2 shows a liquid bottle containing the spraying liquid of the present invention.

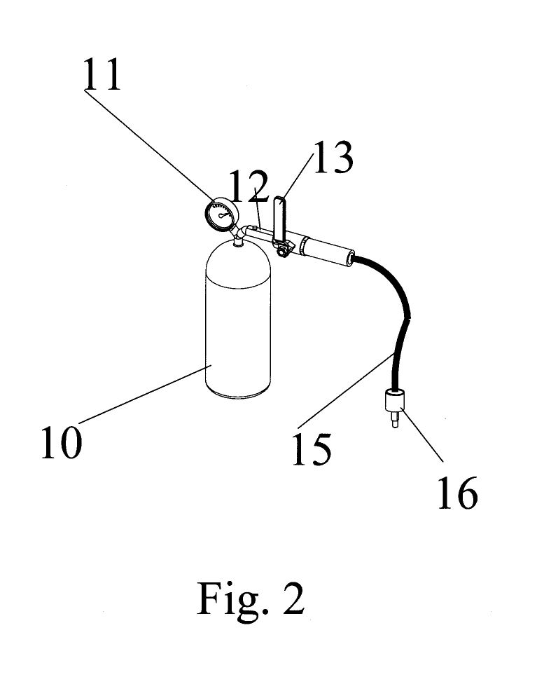

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

A spraying liquid is a solution containing water, cayenne pepper, mustard, natural nano-bacteriostat, emulsion, and potassium sorbate. The components of the liquid of the present invention are described herein:

Contents of cayenne pepper is about 0.1% to 30% of water (weight percentage). Preferable the content of the cayenne pepper is 1% of water.

Contents of mustard is about 0.1% to 30% of water (weight percentage). Preferable the content of the mustard is 1% of water.

The use of the cayenne pepper and cayenne mustard will cause that one breathing them feel very uneasy and thus can not act well for a long period of about 30 minutes. If the cayenne pepper and cayenne mustard contacts eyes, the eyes can not open for a time period of about 30 minutes. Thus the person contacting these components will not act well. This is beneficial in capturing a burglar or a robber.

Contents of natural nano-bacteriostat is about 0.1% to 30% of water (weight percentage). Preferable the content of the natural nano-bacteriostat is 1% of water. The use of natural nano-bacteriostat is to suppress the growth of bacteria so that the spraying liquid of the present invention can sustain for a long time. If no natural nano-bacteriostat is added thereto, the spraying liquid of the present invention is only sustained for about one week.

Contents of emulsion is about 0.1% to 30% of water (weight percentage). Preferable the content of the emulsion is 3% of water. The emulsion can dissolve oily material in the spraying liquid of the present invention.

Contents of potassium sorbate is about 0.1% to 30% of water (weight percentage). Preferable the content of the potassium sorbate is 1% of water. The potassium sorbate is a kind of antiseptic material so that the spraying liquid of the present invention can be preserved for a longer time.

It should be noted that above mentioned spraying liquid of the present invention is harmless to human body so that it can be used safely.

Advantage of the present invention will be described herein. The spraying liquid of the present invention can be sprayed out with a large dispersed area so that the intruder can be sprayed easily as the intruder is moving. That is to say, the spraying liquid of the present invention has a large action area.

Furthermore, the present invention is harmless to human body while it cause the person sprayed by the spraying liquid to be motionless in a period of time which is sufficient for people to catch the person. Thus, the intrudes can not escape from the scene. If an intruder escapes from the scene, it is very difficult to catch it again.

In the present invention, an application of the present invention will be described herein.

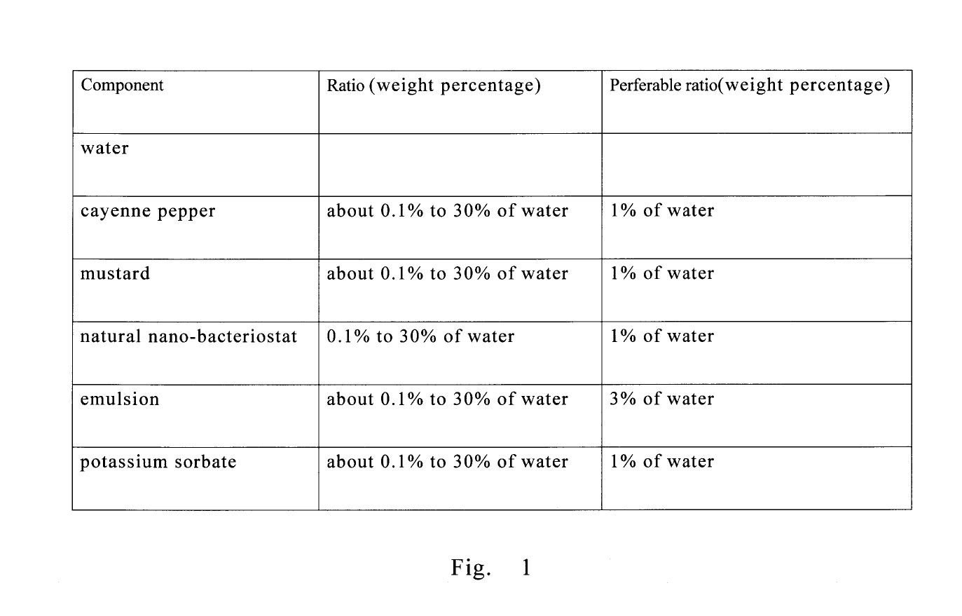

As illustrated in FIG. 1, the spraying liquid of the present invention is placed in a liquid bottle 10 which also contains high pressure gas (such as nitrogen gas). The spraying liquid of the present invention will cause one not to act in certain time period as the liquid is sprayed on the body of a person. The liquid bottle 10 has an opening and a metal tube 12 is extended out from the metal tube 12. Generally, the pressure within the liquid bottle 10 is greater than 10 kgw for providing sufficient pressure to drive the liquid out of the liquid bottle 10 as the opening of the liquid bottle 10 is opened.

A manual operating valve 13 is located at an outer end of metal tube 12.

A liquid guiding tube 15 has a first end connected to the manual operating valve 13 and a second end connected to a spraying head 16 which is detachable.

A pressure sensor 11 is installed at the opening of the bottle 10 for displaying the pressure of an interior of the bottle 10.

In use of the present invention, the user can hold a handle of the bottle and then opening the manual operated valve 13 so that the liquid in the liquid bottle 10 can be sprayed out by the high pressure in the liquid bottle 10 so as to be sprayed to an intruder. As the spraying liquid sprayed upon his (or her) body, he will feel very uneasy for a long time. Thus, as this moment, other people can catch the intruder. Furthermore, the spraying liquid of the present invention only makes those sprayed by the spraying liquid fell very uneasy in a time period which is sufficient to make other people to catch the intrude, while no harm occurs to the intruder.

Advantages of the present invention are that the liquid of the present invention is harmful to human bodies, while a burglar sprayed by the liquid will strongly feel uneasy and thus be motionless for a longer time, thus therefore, other people can capture the burglar easily.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A burglarproof spraying liquid comprising:
water;
cayenne pepper having a content of about 0.1% to 30% of water (weight percentage);
mustard having a content of about 0.1% to 30% of water (weight percentage);
natural nano-bacteriostat having a content of about 0.1% to 30% of water (weight percentage);
emulsion having a content of about 0.1% to 30% of water (weight percentage); and
potassium sorbate having a content of about 0.1% to 30% of water (weight percentage).

2. The spraying liquid as claimed in claim 1, wherein the content of the cayenne pepper is 1% of water.

3. The spraying liquid as claimed in claim 1, wherein the content of the mustard is 1% of water.

4. The spraying liquid as claimed in claim 1, wherein the content of the natural nano-bacteriostat is 1% of water.

5. The spraying liquid as claimed in claim 1, wherein the content of the emulsion is 3% of water.

6. The spraying liquid as claimed in claim 1, wherein the content of the cayenne potassium sorbate is 1% of water.
